(19) United States
(12) Patent Application Publication
(10) Pub. No.: US 2007/0009613 A1
(43) Pub. Date: Jan. 11, 2007

(54) Poisonless sulfur free of noxious ingredients and method for manufacturing the same

(76) Inventor: Bok-Dong Yang, Seoul (KR)

Correspondence Address: LEE, HONG, DEGERMAN, KANG & SCHMADEKA, 801 S. FIGUEROA STREET, 12TH FLOOR, LOS ANGELES, CA, 90017, US

(21) Appl. No.: 11/332,086

(22) Filed: Jan. 13, 2006

(30) Foreign Application Priority Data
Jul. 8, 2005 (KR) 10-2005-0061683

(51) Int. Cl. A61K 36/899; A61K 36/31; A61K 33/04

(52) U.S. Cl. 424/705; 424/750; 424/755

(57) ABSTRACT

The present invention relates to a poisonless sulfur product free of noxious ingredients and a method for manufacturing the poisonless sulfur product free of noxious ingredients, which comprises the steps of: forming a sulfur composition by combining and mixing a 80˜90 wt. % of sulfur, 5˜10 wt. % of barley powder and 5˜10 wt. % of ceramic powder; adding said sulfur composition to radish juice 12˜20 times as much as said sulfur composition before mixing; aging said mixture of sulfur composition with radish juice at 25˜50° C. for 8˜15 hours; and then drying and pulverizing said aged sulfur mixture. Thus, the poisonless sulfur product free of noxious ingredients, low in the thermic property of sulfur, can be safely fed to the livestock directly and the manufacturing method is advantageous in that the number of raw materials for manufacture and the pre-treatment are simple and further the manufacturing work and operation are easy and convenient.

Pursuant to 35 U.S.C. §119(a), this application claims the benefit of earlier filing date and right of priority to Korean Patent Application No. 10-2005-0061683, filed on Jul. 8, 2005, the content of which is hereby incorporated by reference herein in its entirety

### FIELD OF THE INVENTION

The present invention relates to a poisonless sulfur free of noxious ingredients and a method for manufacturing the same and more particularly to a poisonless sulfur product free of noxious ingredients and a method for manufacturing the same, in which the product is obtained by mixing a blend of radish juice, barley power and ceramic power with the sulfur, then aged and dried and can be safely fed to livestock in a great amount.

### BACKGROUND OF THE INVENTION

In the livestock industry, the promotion of the growth of livestock has been tried by using the growth promoters, antibiotics, hormone agents etc. as a means for promoting the livestock. These growth promoters and hormone agents, however, caused the problem that the domestic animals got weak in immunity from chemicals or diseases, and so prone to contract diseases.

Further, a variety of health foods for prevention of the diseases of adult people like corpulence and arterial sclerosis and for healthful life have been developed, as the interest in the health increases with the improvement of the living standard, wherein an interest in the sulfur is recently getting higher as a kind of health food.

The sulfur, which is an element of the oxygen group belonging to the group 6B of the Periodic Table, is reported as having the effect of removing the coldness in the body with its ample thermic property, despite its inherent toxicity, so as to complement the positive or energic nature, and besides treating the ‘deposit of matter’ in heart and the toxin and further relieving the erysipelas, according to the Oriental Medicine Handbook. In addition, according to ancient Chinese literatures, the sulfur, as the principal raw material for the ‘Gold Red’ known as the remarkable medicine, is said to be a panacea for eternal youth and a miracle medicine for rejuvenation. On the other hand, the sulfur is also known, beside the above-described function of complementing energetic nature, to be excellent in counteracting the poison related with the environmental pollution including heavy metals, and agricultural or other chemicals, which is now coming to the front as the causes for cancers and various incurable diseases.

However, the sulfur as a very toxic mineral can be taken internally only after a prescribed process or detoxifying pre-treatment and should be fed, in such a poisonless state, to the livestock or poultry to avoid the 100% lethality.

Accordingly, in the conventional art, the sulfur was detoxified or caused to be less toxic by a known process in which the sulfur was placed in a chinaware vessel exclusively intended for toxic matter, sealed and fired for 48 hours followed by pulverizing. Then, the sulfur product made less toxic was fed as a part of feed stuff to the livestock and poultry, and from these animals, edible meat containing such sulfur was obtained for distribution as a health food.

However, even in the case that the sulfur so attenuated was fed to the livestock or poultry, the lethal rate was not only very high but the limit in the feeding quantity needed also to be set because so-called sweating phenomena was present on the livestock due to the thermic property attributable to the sulfur. It is known that generally the feeding quantity for the attenuated sulfur should not exceed 10 g a day even for the duck which is known as very immune to toxic matter.

Further, according to a report concerning such a conventional method for detoxicating sulfur and a breeding method based on such a detoxicated sulfur, the lethal rate even with such an attenuated sulfur was so high as over 30% so as to necessitate a considerable restriction on the feeding quantity of sulfur, and on the other hand, there was other problem of low retaining effect because even the fed sulfur was excreted with other excrements within nearly 2 hours.

In order to resolve this problem, the present inventor filed a patent application No. 2002-45974 (equivalent to Korean Patent Reg. No. 454574) regarding a poisonless sulfur product free of noxious ingredients and a method for manufacturing the same comprising the steps of: manufacturing a mixed solution by mixing 25˜50 wt. % of oak-tree-cooked water, 0.1˜0.5 wt. % of vinegar, 5˜10 wt. % of barley powder solution, 5˜30 wt. % of turnip juice, 5˜15 wt. % of pine needle juice and the balance of water; mixing the resulting mixed solution with 35˜40 wt. % of sulfur based on the total weight of mixed solution; aging the resulting mixture at 25˜50 ° C. for 2˜3 hours while stirring the mixture; and then drying the aged mixture.

However, for the poisonless sulfur product free of noxious ingredients and a method for manufacturing the same, various raw materials and a tedious pre-treating process are required, so that the manufacturing process is not easy as well, needing further improvement.

### SUMMARY OF THE INVENTION

The present invention was created to resolve the above-described drawbacks. Thus, the object of the invention is to provide a poisonless sulfur product free of noxious ingredients and a method for manufacturing the same, wherein the employed process has the same or more improved detoxifying effect as or than the conventional one so as to be supplied to the livestock at a higher share or greater portion of feed, and the number of raw materials required for manufacturing the product is decreased and the pre-treatment is simplified, with the result that the manufacturing operation is convenient and the process is easy.

The above object is achieved according to a preferred aspect of the invention by a poisonless sulfur product free of noxious ingredients, wherein a sulfur composition resulting from mixing 80˜90 wt. % of sulfur, 5˜10 wt. % of barley powder and 5˜10 wt. % of ceramic powder and radish juice are mixed at the ratio by weight of 1:12˜20, aged at 25˜50° C. for 8˜15 hours and then dried.

Further, the above object is also achieved according to another preferred aspect of the invention by a method for manufacturing poisonless sulfur product free of noxious ingredients, which comprises the steps of: forming a sulfur composition by combining and mixing a 80˜90 wt. % of sulfur, 5˜10 wt. % of barley powder and 5˜10 wt. % of ceramic powder; adding said sulfur composition to radish juice 12˜20 times as much as said sulfur composition before mixing; aging said mixture of sulfur composition with radish juice at 25˜50° C. for 8˜15 hours; and then drying and pulverizing said aged sulfur mixture.

### BRIEF DESCRIPTION OF THE DRAWINGS

FIG. 1 shows a diagram illustrating the result of assay for a pure sulfur by using IR spectrometer(FT-IR),

FIG. 2 shows a diagram illustrating the result of assay for a processed sulfur according to the invention by using IR spectrometer(FT-IR) and

FIG. 3 shows a diagram illustrating the result of thermal assay(TGA) for a processed sulfur according to the invention.

### DETAILED DESCRIPTION OF THE INVENTION

The method for manufacturing poisonless sulfur product free of noxious ingredients according to the invention is described in detail below.

First, the radish, barley and ceramic as the raw materials for use in the invention are prepared, wherein the radish is washed, dried and crushed and then compressed by using a press to result in a radish juice state, and the barley as well as ceramic are also crushed and pulverized to yield a barley powder and a ceramic power.

The representative characteristics of the radish, barley and ceramic used in the invention are as follows.

The radish (Raphanus sativus) is the annual or biennial plant of mustard family, the paoaveraceae order, the dicotyledoneae, wherein the radical leaf is a hairy pinnately compound leaf with the long elliptical shape and the biggest is the apical lobe. The flower stalk branches forth after growing up to about 1 meter and a raceme develops under the flower stalk. Further, pale purple or almost white flowers are in bloom in April or May, arranged in the cross forms and have small peduncles. The calyx is about 7 mm long in a linear long elliptical form. The petal has the form of a wide wedge resembling a reverse egg and is about twice as long as the calyx. There are one pistil and tetradynamous stamens, small glandular bodies being under filaments. The radish nut is about 4˜6 cm long, and contains reddish brown seeds, called as Raphani Semen, but does not burst. The upper part of root is the stem but the boundary is not clear. The root in a fat cylindrical form contains diastase.

The radish is harvested in early winter, stem and leaves are removed and the root thus obtained is washed before use. The sugar components contained in the root are mostly glucose, saccharose and fructose. Besides, in various parts, cumaric acid, caffeic acid, ferulic acid, phenylpyruvic acid, gentisic acid, hydroxybenzoic acid and many kinds of amino acids have been detected. Every single fresh root contains about 7.75 mg of methylthiol and 20 mg of vitamin C but no oxalic acid, so as to be a good calcium source. Further, about 0.41 mg of manganese and 7 mg(on dry base) of boron and a trace of raphanusin were also reported to have been contained. As for medicinal effect, resolving the indigestion, alleviating the feverish phlegm, subduing the excitement, easing the debility, and detoxication are mentioned in the oriental medicine. The cure for indigestion with swelled abdomen, cough with phlegm as well as hoarse voice, hemoptysis, nose-bleeding, diabetes, dysentery and headache is also written.

The seeds of radish, Raphani Semen, may be used for medical purpose. During the ripening time for radish seeds in the summer or autumn, whole stock including seeds are cut, dried under the sun light to obtain the seeds. The seeds contain fatty oil and essential oil which includes methylthiol etc. The fatty oil contains ample quantity of erucic acid, linoleic acid, linolenic acid and ester of glycerinsinapic acid and further includes an antibiotic substance called as raphanin. The relevant medical effect includes the suppression of excitement, ease of breathlessness, digestion of food and alleviation of phlegm. Further remedial effects for the cough with phlegm, indigestion with airway choking, chest trouble with swelled abdomen and diarrhea with after-evacuation discomfort are mentioned as well.

Referring to the records in the Medical Herb Lists etc., the radish juice promotes the digestion, helps relieve the toxicity, favors the five viscera, lightens the body, and makes the skin texture lovely. It also removes the phlegm, stops coughing, cares the hemoptysis, warms the innards, and helps the anemia. There is a record that the green juice can be taken to cure the diarrhea

The radish used for the invention is preferably ordinary radish rather than the turnip for the following reasons.

The desirable important reasons for using the ordinary radish in the invention are that the ordinary radish is easily available at a low price and can provide more ample juice so as to be suited for mass production and further is proper for saving the raw material cost due to the decreased production of waste, although the turnip has about the same sulfur detoxifying sensitivity as the ordinary radish. In other words, the turnip is higher priced and somewhat more difficult to obtain than the ordinary radish to cause a problem in securing a large quantity, and contains the lesser content of water and is harder than the radish, so that it is disadvantageous in manufacturing the radish juice because much waste is produced.

Comparing the composing ratios of the root portion and the leaf portion for the turnip and the ordinary radish, the root portion takes 54.6% and the leaf portion 45.4% for the case of turnip, so that the proportion of leaves is remarkably high compared to the ordinary radish having 89.5% of root portion and 10.5% of leaf portion. This is because for the turnip, the length and size of root are smaller than for the radish even if the leaf part is approximately the same in the shape and size, thus making the composing ratio of leaf portion relatively high.

The barley (Hordeum vulgare var. hexastichon) is a biennial grass of Graminaceae, rice order, monocotyledon, is about 1 meter tall, has hollow cylindrical principal stem, and has widely spaced gnarls. The leaf is approximately in the wide lancet form, 10˜15 mm wide, resists to folding-back, and is light green. The unhulled barley and the rye are distinguished dependent on whether the seeds are attached with hulls or not. Further, the barley is classified as 6-line barley and 4-line barley dependent on the number of kernel lines on a spike. The barley in which the lemmas and paleas are firmly attached to seeds and not fallen out after the ripening is called the unhulled barley, while the barley in which the hulls are easily stripped off is called as the rye. The barley is also broken down into the hexagonal and tetragonal barley in accordance with the form of the cross section of spike. The barley is used as foodstuffs, feed stuffs and industrial raw materials (brewed beer, malts etc.), as is well known.

The ingredients of the barley include the fibrous matters, starch, ash, nitrogen compounds, vitamin B and C, minerals, inorganic salts, fats as well as lecitin, maltose and saccharose. The barley contains higher content of protein than the rice, is ample in essential amino acids, and so is effective in preventing the aging of blood vessels, preventing the beriberi, protecting the stomach and preventing geriatric diseases. Particularly, the barley contains a great deal of water-soluble edible fiber, i.e. beta glucan acting the role of decreasing cholesterol which causes the heart diseases like arterial sclerosis, hypertension, diabetes etc. In addition, the barley is excellent in stability due to the property of cooling the heat of body.

The ceramics which is composed of inorganic non-metallic material high-temperature teated after moulding refers generally to ceramic products. The chinawares in the form of ordinary household articles are included as a matter of course. The porous unglazed stonewares and some of glazed earthenwares are fired at not so high a temperature but the chinawares etc. are fired at as high as about 1,200˜1,400° C., wherein the base is dense, translucent and glassy.

Whereas the main components of ceramics are Si, Al, Mg and Na, the thermal, mechanical and optical properties of ceramics would chiefly vary dependent on the kind, amount and arrangement of the composing atoms.

As the raw material for the ceramic powders used in the invention, SiO₂, Al₂O₃, Fe₂O₃, CaO, MgO, Na₂O, K₂O, TiO₂, P₂O₅, B₂O₃, SO₃ and H₂O, these oxides added rare-earth elements, and besides the sulfides, nitrides, carbides and silicides can be mentioned.

Generally, the ceramics has a variety of characteristics such as thermal stability, high-temperature strength, high elasticity, high hardness, soft magnetism, hard magnetism, insulating property, piezoelectricity, pyroelectricity, ionic conductivity, optical conductivity, resistance calorificaftion, thermal electron emission, fluorescence-luminescence, IR permeability, high transparence, electrochromaticity. The thermal stability among those listed properties, i.e. the excellent heat resistance is used advantageously in the invention for decreasing thermal conductivity and blocking heat generation.

The role of ceramic powder in the invention is detoxicating the sulfur product and decreasing thermic property of the sulfur and besides preventing the mixture, during the steps of mixing with the sulfur, radish juice and barley powder, aging and drying, from aggregating or hardening like dough, so as to crisp and easily dryable.

5˜10 wt. % of the barley powder and 5˜10 wt. % of the ceramic powder(e.g. water-soluble) so prepared as in the above are blended with 80˜90 wt. % of the sulfur and mixed to manufacture the sulfur composition. The mixing ratio of the sulfur, barley and ceramic powder, composing the above-described composition, is that, as determined as satisfactory for detoxifying the sulfur through many runs of repeated tests by the present inventor, wherein the use of the above three components over or below the prescribed range followed by mixing and processing would lead to the prolonged detoxifying period or unsatisfactory detoxification result.

Adding said sulfur composition to a radish juice 12˜20 times as much as said sulfur composition results in the desired mixture.

Regarding the addition ratio of the radish juice, the noxious substance of sulfur is not effectively removed and the disgusting smell of sulfur is given out in the finished feed stuff, when the radish juice is mixed below 12 times the quantity of sulfur composition. On the other hand, when the radish juice is mixed over 20 times the quantity of sulfur composition, the noxious substance of sulfur is removed enough but uneconomical. Accordingly, the radish juice is preferably added in 12˜20 times the quantity of sulfur composition.

To facilitate the solving of the powdery sulfur in the radish juice mixed with the barley powder and ceramic powder, first the barley powder and ceramic powder are added to the radish juice followed by mixing to result in a mixture to which the sulfur put in a fine-meshed net is added in a rubbing manner so as to be solved, wherein the amounts of the four components are respectively used to fulfill the above-proposed ratio.

The mixture of sulfur composition and radish juice is subjected to an ageing process at 25˜50° C. for 8˜15 hours, wherein stirring is preferably accompanied to achieve a uniform and rapid aging.

In the case of aging below 25° C., the aging becomes incomplete and requires a longer time so that the manufacturing process for poisonless sulfur product according to the invention becomes uneconomical. To the contrary, if the aging temperature exceeds 50° C., the components of the sulfur composition may experience a certain change. Thus, the appropriate temperature ranges from 25° C. to 50° C. Most preferably, the aging temperatures in the winter and summer are 25˜35° C. and 30˜50° C. respectively.

On the other hand, although the sulfur detoxifying effect gets better as the aging time for sulfur mixture increases, the aging for more than 15 hours simply results in the increased cost without the appreciable improvement in the detoxification result and the taste of feed stuff, When the aging time is less than 8 hours, however, the aging effect gets insufficient. As the result, the aging for 8˜15 hours is desirable.

The above-described aging time and the aging temperature used in the invention are approximately in the relation of inverse proportion. While the aging temperature of 50° C. needs only the aging time of 8 hours for a sufficient detoxifying effect, the aging temperature of 25° C. would need the aging time of 15 hours for the same sufficient detoxifying effect.

The aged mixture as described above is dried to provide the finished sulfur product free of noxious components according to the invention. In that drying step, the aged mixture is left to be cooled for 30 minutes˜1 hour to allow the precipitate to settle to the bottom, letting the clear liquid float on the top. After that separation, the clear liquid is decanted out and the settled precipitate is caused to be dried to the completion in the shade. A variety of drying methods may be used for the above drying process but the use of vacuum driers or the like would be proper.

The dried sulfur product free of noxious components is then pulverized in a pulverizer so as to provide fine powder with the fineness corresponding to over 350-mesh.

The sulfur product free of noxious components according to the invention so manufactured can be either in conjunction with a conventional blended livestock feed or alone as such to the livestock, poultry and cultured fish.

Furthermore, the sulfur product free of noxious components according to the invention can be applied to various fields like the cosmetic, medicine, purified sulfur for internal use etc., beside the feed for livestock.

### EXAMPLE 1

Poisonless Sulfur Product Free Of Noxious Ingredients

Radishes were washed and made free of water to dryness and then crushed in multi-stages and pressed by using a press to be in radish juice state. Barley grains and ceramics were also pulverized to become powder, whereby the barley powder and ceramic powder were prepared.

5 wt. % of the barley powder and 5 wt. % of the ceramic powder so prepared as above were blended with 90 wt. % of the sulfur, followed by mixing, so as to provide a sulfur composition.

Adding said sulfur composition to a radish juice in 20 times the quantity of said sulfur composition resulted in a sulfur mixture.

The thus-obtained sulfur mixture was put in a tank maintained at 35° C., and aged for 10 hours under agitation.

The agitated sulfur mixture was left free at normal temperature. After 1 hour, the precipitate was settled to the bottom while the clear liquid floated above, so that the upper and lower sections were formed. The clear liquid was decanted out and the settled precipitate was caused to be dried to the completion in the shade.

The dried precipitate was then pulverized in a pulverizer to 350-mesh powder, so as to provide a poisonless sulfur free of noxious ingredients according to the invention.

### TEST EXAMPLE 1

Comparison of a General Sulfur with an Inventive Poisonless Sulfur.

A poisonless sulfur product free of noxious ingredients according to the invention so finished as described above was tested for x-ray photographing and measurement of density, whereby the result of assay is denoted in Table 1.

TABLE 1

| Sample | XRD*¹ | Density*² |
|---|---|---|
| General | α-S as main mineral | 2.18 |
| Sulfur feed according to the inv. | α-S as main mineral | 2.13 |

*¹Instrment manuf. by MAC Science used for XRD test.
*²Instrment manuf. by Micromeritics(U.S.A) Accupyc ™ 1330 used for Density test.

As appreciated from the above table, the main mineral was α-S for both the inventive sulfur and general sulfur, indicating no external change following the manufacturing process, and the density was shown to be only a little lower relative to the general sulfur. The easily available crude sulfur may be usable for the raw material of the invention.

### TEST EXAMPLE 2

Noxiousness Assay

For noxious assay, the ionic assay(ICP-MS) and the elemental assay(EA) following pre-treatment were employed.

1. Ionic Assay(ICP-MS)

In order to analyze the components, the samples were first treated with 70% high conc. nitric acid solution and then subjected to the ion-chromatography to separate components and subjected to the qualitative analysis through mass spectrometer.

The result of the mass analysis conducted with reference to the standard substances after confirming the resolving ability(peaks) of respective components in primary qualitative analysis is shown in the following Table 2.

TABLE 2
(ppm)

| | Assay items | | | | | |
|---|---|---|---|---|---|---|
| | Pb | Cd | Cr | Hg | Se | As |
| Ass. values | Trace | Trace | Trace | Trace | 7.42(*1) | Trace(*2) |

(*1)somewhat higher value due to interference by peaks of components
(*2)Trace means less than 3 ppm, neglectable as within error limits.

2. Elemental Assay(EA)

The elemental assay(EA) conducted while burning the sulfur under nitrogen atmosphere by using an element analyzer was run twice. The result is shown in Table 3.

TABLE 3
(wt. %)

| | Ass. items | | |
|---|---|---|---|
| | Sulfur content | Carbon content | Others |
| Ass. value 1 | 98.58 | 1.30 | |
| Ass. value 2 | 98.76 | 1.39 | |
| Average assay value | 98.67 | 1.34 | |

As shown above, from the shown result of noxious component assay which can serve as the basis for assessment in applying the processed sulfur according to the invention to the noxiousness-related livestock feed, the processed sulfur according to the invention may be judged to have no problem on the base of the aspect related with the purity of sulfur product for using the inventive processed sulfur product as feed stuff for livestock.

### TEST EXAMPLE 3

Assay for Organic Property

1. Extraction Test with Organic Solvent

This test is intended to confirm the organic ingredients contained within the processed or detoxified sulfur product according to the invention if any, on the ground that any organic matter present in the sulfur product would be dissolved in the organic solvent.

A pure sulfur and the poisonless sulfur according to the invention were subjected to solvent extraction test on the same conditions for 24 hours with methyl alcohol as the organic solvent and the resultant organic solvent was examined for the presence of any organic components by the IR spectrometer(FT-IR), wherein the qualitative confirmation experiment showed no characteristic peaks indicative of organic matters over the all wavelengths( despite the barley (or radish) for instance).

The pure sulfur as the control contained no organic matter as a matter of course and the sample given for test likewise contained no organic matter.

2. Qualification Confirmation Test

This test was conducted as a further method of confirming any possible organic matter in samples, by using the IR spectrometer(FT-IR).

The samples of the inventive sulfur and the pure sulfur together with KBr were first prepared to solutions of proper concentrations by using a diluent because the samples for test are solid, before testing for comparison at the same conditions.

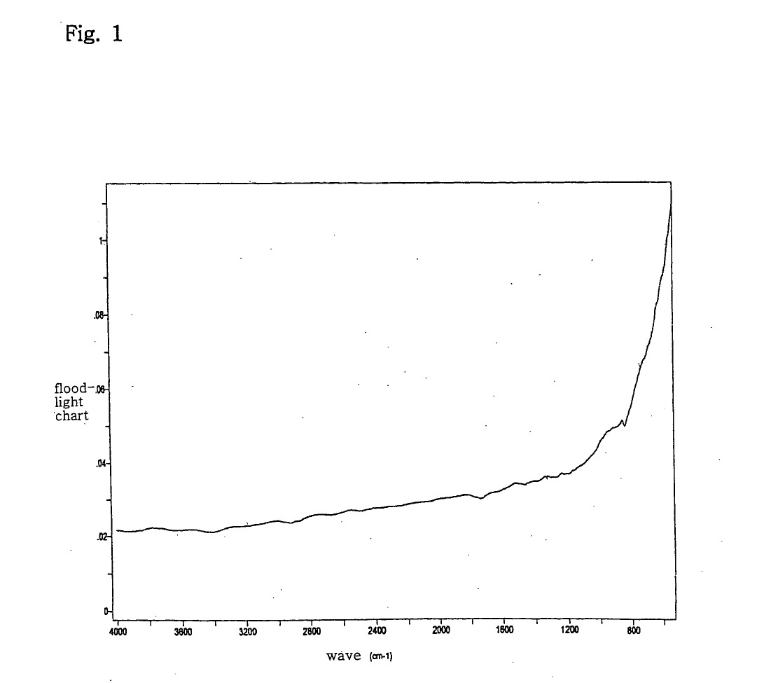

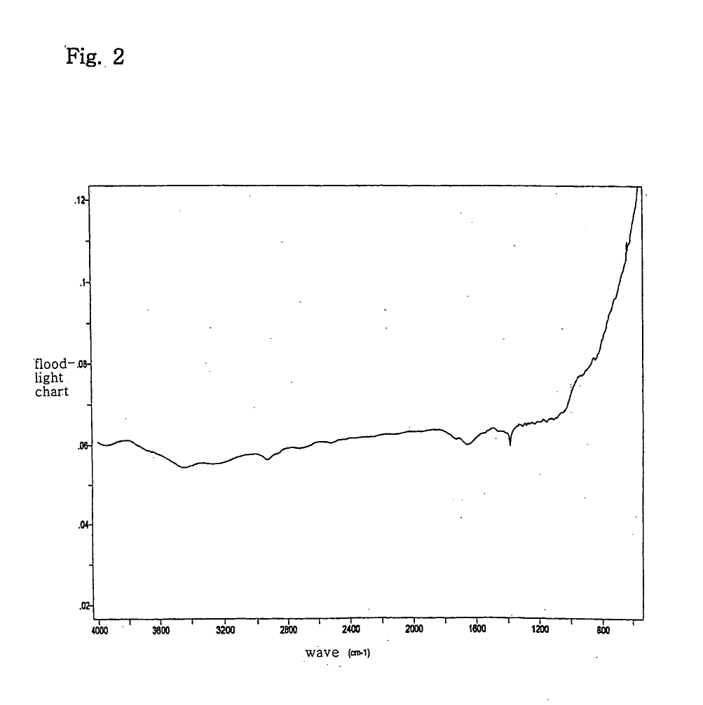

The result of assay by using an IR spectrometer(FT-IR) is shown in FIGS. 1 and 2, wherein FIG. 1 is for the invention and FIG. 2 for the pure sulfur.

As apparent from the figures, the both samples showed about the same behavior and no peaks characteristic of organic matters were detected.

3. Thermal Assay(TGA)

This test was conducted to see the variance in weight when burning the processed sulfur sample according to the invention, because two steps of curves would appear due to different ignition temperatures if the sample is composed of a mineral and an organic matter. This TGA is frequently applied to unknown solid samples to determine whether the samples are composed of a pure substance.

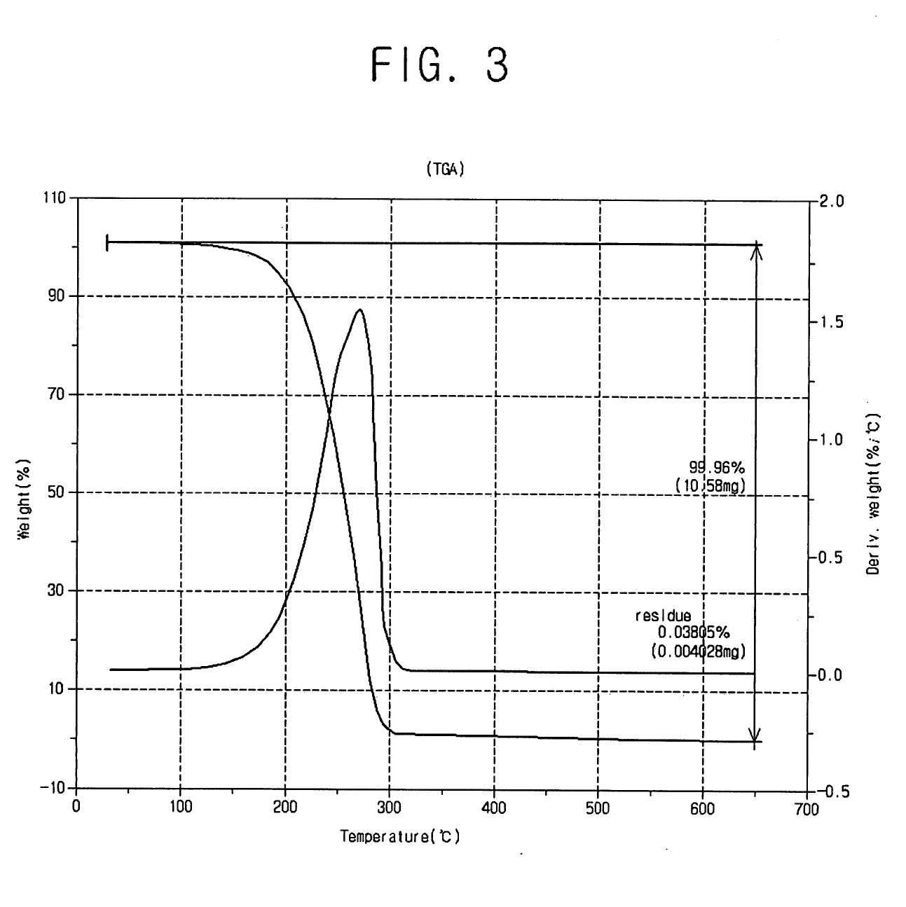

The assay result is shown in FIG. 3.

The result of thermal test shows the curve behavior characteristic of combustion of a pure substance. Accordingly, the processed sulfur according to the invention contained no organic substance like the pure sulfur, so as to be suited e.g. for an organic matter-free pure sulfur application.

### TEST EXAMPLE 4

Assay for Crystal(XRD)

Additionally, the assaying for comparison through the crystal analysis was conducted to examine any possible change of the sulfur structure in the course of manufacturing the detoxified sulfur, in consideration of the characteristic effect of the detoxified sulfur according to the invention, particularly as livestock feed.

The assay of crystal for finding any change in the structure of the inventive detoxified sulfur and the pure sulfur was generally intended to find any differentiated transition between a crystal state and an amorphous state for both samples.

The crystal assay, which was to confirm any change in crystals based on the changed behavior of reflected ray after the X ray was irradiated onto the sample, showed about the same result for the both samples, meaning no apparent or detectable change after the processing for the case of the invention.

### TEST EXAMPLE 5

Application of Detoxified Sulfur

The poisonless sulfur free of noxious ingredients according to the invention manufactured as in EXAMPLE1 was supplied as single feed, without mixing with any other feed stuffs, to oxen, pigs, fowls and cultured fishes for 3 months.

As the result, the oxen, pigs, fowls and cultured fishes which took the poisonless sulfur free of noxious ingredients according to the invention exhibited the sustaining medicinal sign in the intestine after 1 month on the average. After 2 months, the sustaining medicinal sign was uniformly extended to the flesh and blood. After 2 months on and thereafter, even the bone had a smack of sulfur, whereby strengthened bones seemed evident.

The most important characteristic was that the poisonless sulfur free of noxious ingredients according to the invention, differently from the conventional sulfur free of noxious ingredients, could be fed to the livestock in a sufficient amount, without a limit.

The poisonless sulfur free of noxious ingredients according to the invention is enough freed of the thermic toxicity of sulfur to be low in the thermic toxicity of sulfur, with the result that the inventive product has the advantage of being able to feed alone or in combination with other feed to the livestock.

Particularly, the poisonless sulfur free of noxious ingredients according to the invention has the economic effect of low cost because the number of raw materials for manufacture and the pre-treatment are simple and further the manufacturing work and operation are easy and convenient.

1. A poisonless sulfur product free of noxious ingredients, wherein a sulfur composition resulting from mixing 80˜90 wt. % of sulfur, 5˜10 wt. % of barley powder and 5˜10 wt. % of ceramic powder and radish juice are mixed at the ratio by weight of 1: 12˜20, aged at 25˜50° C. for 8˜15 hours and then dried.

2. A method for manufacturing poisonless sulfur product free of noxious ingredients, which comprises the steps of:
forming a sulfur composition by combining and mixing a 80˜90 wt. % of sulfur, 5˜10 wt. % of barley powder and 5˜10 wt. % of ceramic powder;
adding said sulfur composition to radish juice 12˜20 times as much as said sulfur composition before mixing;
aging said mixture of sulfur composition with radish juice at 25˜50° C. for 8˜15 hours; and then
drying and pulverizing said aged sulfur mixture.
